(19) United States
(12) Patent Application Publication
(10) Pub. No.: US 2008/0207907 A1
(43) Pub. Date: Aug. 28, 2008

(54) Process For Pd-Catalysed C-N Coupling In Specific Solvent Systems

(75) Inventors: Björn Schlummer, Bonn (DE); Ulrich Scholz, Mulheim an der Ruhr (DE); Ian Smith, Hertfordshire (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Bruxelles (BE)

(21) Appl. No.: 11/718,898

(22) PCT Filed: Nov. 23, 2005
(86) PCT No.: PCT/EP05/12509

§ 371 Date: Dec. 3, 2007

(30) Foreign Application Priority Data
Nov. 24, 2004 (DE) 10 2004 056 820.0
Nov. 24, 2004 (DE) 10 2004 056 821.9

(51) Int. Cl. C07D 471/00

(52) U.S. Cl. 546/114; 546/115; 546/113

(57) ABSTRACT

The invention relates to a novel process for the Pd catalysed formation of C—N bonds between an aryl halide or an aryloxysulphonyl compound and an amine in specific solvent systems, and also to the use of these solvent systems for C—N couplings.

The invention relates to a novel process for the Pd-catalysed formation of C—N bonds between an aryl halide or an aryloxysulphonyl compound and an amine in specific solvent systems, and also to the use of these solvent systems for C—N couplings.

The formation, catalysed by palladium compounds, of a C—N bond between an aryl halide and an amine, which builds on studies by Buchwald and Hartwig, has gained great significance in the industrial synthesis of starting materials for the production of commodity and specialty chemicals which find use in applications including those in the agrochemical and pharmaceutical industry.

The technique itself is summarized in a multitude of patents and review articles.

For instance, Buchwald in review articles such as B. H. Yang, S. L. Buchwald, J. Organomet. Chem. 1999, 576, 125-146 and Muci, A. R., Buchwald, S. L., Top. Curr. Chem. 2002, 219, 131-209 reports on the palladium-catalysed amination of aryl halides. It is stated that monodentate bis-alkyl-substituted phosphines based on a basic biphenyl structure are often found to be suitable ligands for the catalysis of transition metal-catalysed coupling.

In several review articles such as J. F. Hartwig in Modern Arene Chemistry 2002, 107-168 and J. F. Hartwig in Handbook of Organopalladium Chemistry for Organopalladium Chemistry of Organic Synthesis 2002, 1, 1051-1096, Hartwig likewise reports on the state of the art in the field of palladium-catalysed C—N bond formation.

Furthermore, Buchwald reports, in several patents, on the use of biphenyl-based ligand systems. WO-A 2000/002887 and U.S. Pat. No. 6,307,087 claim the preparation of arylamine compounds by reaction of an amine with an aromatic compound using palladium complexes.

Newer biphenyl-based ligands, in particular 2,4,6-triisopropyl-2′-biscyclohexylphosphinobiphenyl, are described by Buchwald in X. Huang, K. W. Anderson, D. Zim, L. Jiang, A. Klapars, S. L. Buchwald, J. Am. Chem. Soc. 2003, 125, 6653-6655 and E. R. Strieter, D. G. Blackmond, S. L. Buchwald, J. Am. Chem. Soc. 2003, 125, 13978-13980.

The addition of polar solvents such as tert-butanol or water is described in R. A. Singer, S. Caron, R. E. McDermott, P. Arpin, N. M. Do, Synthesis 2003, 11, 1727-1731, M. H. Ali, S. L. Buchwald, J. Org. Chem. 2001, 66, 2560-2565, R. Kuwano, M. Utsunomiya, J. F. Hartwig, J. Org. Chem. 2002, 67, 6479-6486, J. Yin, M. M. Zhao, M. A. Huffman, J. M. McNamara, Org. Lett. 2002, 4, 20, 3481-3484 and S. R. Stauffer, J. F. Hartwig, J. Am. Chem. Soc. 2003, 125, 6977-6985.

In many of the reactions described, one of the fundamental problems is a slowing in the conversion of the substrate during the course of the reaction, which can lead to the catalytic reaction stopping completely.

As potential reasons for this, a decrease in the activity of the catalyst system and also depletion of available substrate or base by adsorption of the reaction products on the base or substrate surface are discussed.

There is thus still a need for a process for forming C—N bonds, in which the above-detailed disadvantage of incomplete conversion is remedied or at least distinctly reduced.

The object on which the present invention is based is thus to discover a process for the formation of C—N bonds in which the conversion of the substrate is enhanced compared to known processes. In particular, it is a further object to discover such a process for the reaction of aryl halides or aryloxysulphonyl compounds with an amine.

It has been found that, surprisingly, such an increased conversion is achieved with the use of specific solvent systems comprising at least two, preferably more than two solvents selected from ethers, cyclic ethers, tertiary amines, aromatic hydrocarbons, alcohols and water. An increase in the conversion of the amine used as the substrate by such a combination of two or preferably more than two solvents has to date not been described in the literature.

The present invention thus provides for the use of such a solvent system comprising at least two, preferably more than two solvents selected from ethers, cyclic ethers, tertiary amines, aromatic hydrocarbons, alcohols and water for C—N couplings.

Very particular preference is given to the use of a solvent system comprising at least three or more solvents selected from ethers, cyclic ethers, tertiary amines, aromatic hydrocarbons, alcohols and water. In a preferred embodiment, a solvent system comprising four solvents selected from ethers, cyclic ethers, tertiary amines, aromatic hydrocarbons, alcohols and water is used.

Examples of suitable solvents in the context of the invention are listed below. Suitable ethers are, for example, dialkyl ethers, for example dimethyl ether, diethyl ether, diisopropyl ether, methyl t-butyl ether (MTBE), isopropyl t-butyl ether, diaryl ethers or polyethers, e.g. 1,2-dimethoxyethane (DME), the “poly” being understood in the sense of two or more. Suitable cyclic ethers are, for example, tetrahydrofuran (THF), methyltetrahydrofuran (methyl-THF), tetrahydropyran (THP) or dioxane. Suitable tertiary amines are, for example, trialkylamines, triarylamines or mixed aliphatic-aromatic tertiary amines, for example triethylamine, tri-n-propylamine, triisopropylamine, tri-n-butylamine (TBA) or triphenylamine. Suitable aromatic hydrocarbons are, for example, toluene, xylenes, anisole, veratrole, benzene or chlorobenzene. Suitable alcohols are, for example, methanol, ethanol, n-propanol, isopropyl alcohol, n- or tert-butanol, n-pentanol, n-hexanol, n-, sec- or tert-amyl alcohol.

The solvents specified may be present in the solvent system in any mixing ratios. Preferably, water is present if appropriate in the solvent system in an amount of 20% by volume or less, preferably 5% by volume or less, more preferably 3% by volume or less. An ether is present if appropriate in the solvent system preferably in an amount of 50% by volume or less, preferably 30% by volume or less, more preferably 25% by volume or less. A cyclic ether is present if appropriate in the solvent system preferably in an amount of 75% by volume or less, preferably 50% by volume or less, more preferably 25% by volume or less. A tertiary amine is present if appropriate in the solvent system preferably in an amount of 95% by volume or less, preferably 85% by volume or less, more preferably 75% by volume or less. An aromatic hydrocarbon is present if appropriate in the solvent system preferably in an amount of 95% by volume or less, preferably 85% by volume or less, more preferably 75% by volume or less. An alcohol is present if appropriate in the solvent system preferably in an amount of 75% by volume or less, preferably 50% by volume or less, more preferably 25% by volume or less.

The proportions of the individual solvents in the solvent system add up to 100% by volume.

A preferred embodiment is the use of a solvent system comprising at least two solvents selected from tri-n-butylamine, 1,2-dimethoxyethane, tetrahydrofuran and water. In this solvent system, very particular preference is given to using 1.5% by volume or less of water, 15% by volume or less of tetrahydrofuran, 25% by volume or less of 1,2-dimethoxyethane and sufficient tri-n-butylamine that the proportions added together give 100% by volume. An example of such a very particularly preferred ratio is a volume ratio of 71:18:19:1% by volume of tri-n-butylamine:1,2-dimethoxyethane:tetrahydrofuran:water. An especially more preferred ratio is a volume ratio of 70:18:11:1% by volume of tri-n-butylamine:1,2-dimethoxyethane:tetrahydrofuran:water.

The solvent systems detailed above are suitable preferentially for Pd-catalysed C—N couplings in which an aryl halide or an aryloxysulphonyl compound is reacted with an amine. In particular, compounds of the general formula (II)

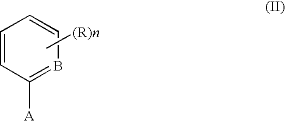

in which
R is each independently H, optionally substituted C₁-C₁₈-alkyl, preferably C₁-C₆-alkyl, optionally substituted C₁-C₁₈-alkoxy, preferably C₁-C₆-alkoxy, optionally substituted C₄-C₂₄-aryl, preferably C₆-C₂₄-aryl, optionally substituted C₅-C₁₈-arylalkyl, halogen preferably fluorine, pseudohalogen, OH, NO₂ or COR^{x} in which R^{x} is OH, OR^{y} or NR^{y}R^{z}, where R^{y} and R^{z} are each independently optionally substituted C₁-C₁₈-alkyl, preferably C₁-C₆-alkyl, optionally substituted C₄-C₂₄-aryl, preferably C₆-C₂₄-aryl, or optionally substituted C₅-C₁₈-arylalkyl,
n is 0 or an integer of 1 to 5 and
A is halogen or —OR⁶ in which R⁶ is optionally substituted C₁-C₁₈-alkylsulphonyl, preferably C₁-C₆-alkylsulphonyl, or optionally substituted C₄-C₂₄-arylsulphonyl, preferably C₆-C₂₄-arylsulphonyl
B is CH or N
   are coupled with compounds of the general formula (III)

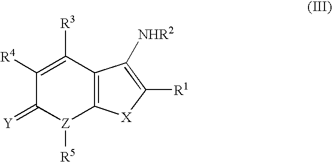

in which
R² is H, optionally substituted C₁-C₁₈-alkyl, preferably C₁-C₆-alkyl, optionally substituted C₄-C₂₄-aryl, preferably C₆-C₂₄-aryl, or optionally substituted C₅-C₁₈-arylalkyl,
X is O, S, NH or CH₂,
Y is O or S,
z is N or CH,
R¹, R³, R⁴ are each independently H, optionally substituted C₁-C₁₈-alkyl, preferably C₁-C₆-alkyl, optionally substituted C₁-C₁₈-alkoxy, preferably C₁-C₆-alkoxy, optionally substituted C₄-C₂₄-aryl, preferably C₆-C₂₄-aryl, optionally substituted C₅-C₁₈-arylalkyl, halogen, optionally substituted C₁-C₁₈-alkylcarbonyl, preferably C₁-C₆-alkylcarbonyl, optionally substituted C₄-C₂₄-arylcarbonyl, preferably C₆-C₂₄-arylcarbonyl, —COOR⁷, pseudohalogen, OH, NO₂, COR^{x} in which R^{x} is OH, OR^{y}, NR^{y}R^{z}, or NHet, where R^{y} and R^{z} are each independently optionally substituted C₁-C₁₈-alkyl, preferably C₁-C₆-alkyl, optionally substituted C₄-C₂₄-aryl, preferably C₆-C₂₄-aryl, or optionally substituted C₅-C₁₈-arylalkyl, and NHet is an optionally substituted 4- to 6-membered heterocycloalkyl group attached through a nitrogen atom to the group CO,
R⁵ is H, optionally substituted C₁-C₁₈-alkyl, preferably C₁-C₆-alkyl, optionally substituted C₄-C₂₄-aryl, preferably C₆-C₂₄-aryl, or optionally substituted C₅-C₁₈-arylalkyl,
R⁷ is an optionally substituted aryloxyaryl, alkanoyloxyalkyl or aroyloxyalkyl,
   to give compounds of the general formula (I)

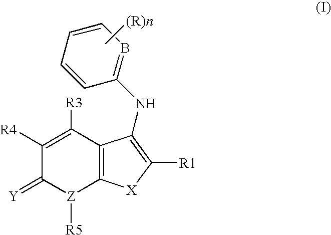

in which R¹, R³ to R⁵, X, Y and Z are each as defined for the general formula (III) and B, R and n are as defined for general formula (II).

The present invention thus further provides a process for preparing compounds of the general formula (I)

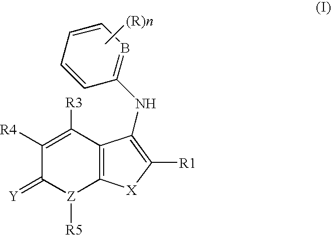

in which
X is O, S, NH or CH₂,
Y is O or S,
z is N or CH,
R¹, R³, R⁴ are each independently H, optionally substituted C₁-C₁₈-alkyl, preferably C₁-C₆-alkyl, optionally substituted
C₁-C₁₈-alkoxy, preferably C₁-C₆-alkoxy, optionally substituted C₄-C₂₄-aryl, preferably C₆-C₂₄-aryl, optionally substituted C₅-C₁₈-arylalkyl, halogen, optionally substituted C₁-C₁₈-alkylcarbonyl, preferably C₁-C₆-alkylcarbonyl, optionally substituted C₄-C₂₄-arylcarbonyl, preferably C₆-C₂₄-arylcarbonyl, —COOR⁷, pseudohalogen, OH, NO₂, COR^{x} in which R^{x} is OH, OR^{y}, NR^{y}R^{z}, or NHet where R^{y} and R^{z} are each independently optionally substituted C₁-C₁₈-alkyl, preferably C₁-C₆-alkyl, optionally substituted C₄-C₂₄-aryl, preferably C₆-C₂₄-aryl, or optionally substituted C₅-C₁₈-arylalkyl, and NHet is an optionally substituted 4- to 6-membered heterocycloalkyl group attached through a nitrogen atom to the group CO,
R⁵ is H, optionally substituted C₁-C₁₈-alkyl, preferably C₁-C₆-alkyl, optionally substituted C₄-C₂₄-aryl, preferably C₆-C₂₄-aryl, or optionally substituted C₅-C₁₈-arylalkyl,
R⁷ is an optionally substituted aryloxyaryl, alkanoyloxyalkyl or aroyloxyalkyl,
R is each independently H, optionally substituted C₁-C₁₈-alkyl, preferably C₁-C₆-alkyl, optionally substituted C₁-C₁₈-alkoxy, preferably C₁-C₆-alkoxy, optionally substituted C₄-C₂₄-aryl, preferably C₆-C₂₄-aryl, optionally substituted C₅-C₁₈-arylalkyl, halogen preferably fluorine, pseudohalogen, OH, NO₂ or COR^{x} in which R^{x} is OH, OR^{y} or NR^{y}R^{z}, where R^{y} and R^{z} are each independently optionally substituted C₁-C₁₈-alkyl, preferably C₁-C₆-alkyl, optionally substituted C₄-C₂₄-aryl, preferably C₆-C₂₄-aryl, or optionally substituted C₅-C₁₈-arylalkyl,
B is CH or N, and
n is 0 or an integer of 1 to 5,
   by reacting compounds of the general formula (II)

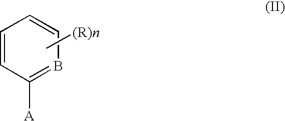

in which
A is halogen or —OR⁶ in which R⁶ is optionally substituted C₁-C₁₈-alkylsulphonyl, preferably C₁-C₆-alkylsulphonyl, or optionally substituted C₄-C₂₄-arylsulphonyl, preferably C₆-C₂₄-arylsulphonyl,
   and R, B and n are each as defined for the general formula (I),
   with compounds of the general formula (III)

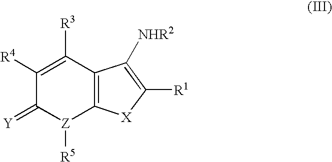

in which
R² is H, optionally substituted C₁-C₁₈-alkyl, preferably C₁-C₆-alkyl, optionally substituted C₄-C₂₄-aryl, preferably C₆-C₂₄-aryl, or optionally substituted C₅-C₁₈-arylalkyl, and
R¹, R³ to R⁵, X, Y and Z are each as defined for the general formula (I),
   in the presence of a catalyst system comprising at least one palladium precursor, at least one ligand and at least one base, wherein the reaction is carried out in a solvent system comprising at least two, preferably more than two solvents selected from ethers, cyclic ethers, tertiary amines, aromatic hydrocarbons, alcohols and water.

For the areas of preference for the composition of the solvent system, those already detailed above apply analogously.

Alkyl or alkoxy are each independently a linear, cyclic, branched or unbranched alkyl or alkoxy radical. The same applies to the nonaromatic moiety of an arylalkyl radical and also to alkyl or alkoxy constituents of more complex groups, for example alkylcarbonyl or alkylsulphonyl radicals.

C₁-C₆-alkyl is, for example, methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl or 1-ethyl-2-methylpropyl, C₁-C₁₈-alkyl is, for example, n-heptyl and n-octyl, pinacoyl, adamantyl, the isomeric menthyls, n-nonyl, n-decyl, n-dodecyl, n-tridecyl, n-tetradecyl, n-hexadecyl, n-octadecyl or stearyl.

C₁-C₁₈-alkoxy is, for example, the alkoxy groups corresponding to the above alkyl groups, for example methoxy, ethoxy, n-propoxy, isopropoxy, n-butoxy, sec-butoxy, tert-butoxy, etc.

Aryl is in each case independently an aromatic radical having 4 to 24 skeleton carbon atoms, in which no, one, two or three skeleton carbon atoms per cycle, but at least one skeleton carbon atom in the entire molecule, may be replaced by heteroatoms selected from the group of nitrogen, sulphur or oxygen, but is preferably a carbocyclic aromatic radical having 6 to 24 skeleton carbon atoms. The same applies to the aromatic moiety of an arylalkyl radical, and also to aryl constituents of more complex groups, for example arylcarbonyl or arylsulphonyl radicals.

Examples of C₆-C₂₄-aryl are phenyl, o-, p-, m-tolyl, 2,6-difluorophenyl, naphthyl, phenanthrenyl, anthracenyl or fluorenyl; examples of heteroaromatic C₄-C₂₄-aryl in which no, one, two or three skeleton carbon atoms per cycle, but at least one skeleton carbon atom in the entire molecule, may be substituted by heteroatoms selected from the group of nitrogen, sulphur or oxygen are, for example, pyridyl, pyridyl N-oxide, pyrimidyl, pyridazinyl, pyrazinyl, thienyl, furyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl, benzofuranyl or dibenzofuranyl.

Arylalkyl is in each case independently a straight-chain, cyclic, branched or unbranched alkyl radical as defined above which may be substituted singly, multiply or fully by aryl radicals as defined above.

C₅-C₁₈-arylalkyl is, for example, benzyl or (R)- or (S)-1-phenylethyl.

The term “arylcarbonyl” as used herein represents a group of formula —CO-aryl wherein aryl is as defined herein.

The term “alkylcarbonyl” as used herein represents a group of formula —CO-alkyl wherein alkyl is as defined herein.

The term “aryloxyalkyl” is intended to refer to a straight or branched alkyl group, as defined herein, wherein a terminal hydrogen atom is replace with an aryl-O— group, where the aryl group is as defined herein.

The term “alkanoyloxyalkyl” refers to a straight or branched alkyl group, as defined herein, wherein a terminal hydrogen atom is replaced with an alkyl-C(O)O— group, where the alkyl group is as defined herein.

The term “aroyloxyalkyl” refers to a straight or branched alkyl group, as defined herein, wherein a terminal hydrogen atoms is replaced with an aryl-C(O)O— group, where the aryl group is as defined herein.

The term “heterocycloalkyl group” refers to an optionally substituted non-aromatic 4- to 6-membered saturated monocyclic hydrocarbon ring system containing one or two heteroatoms selected from O, N and S.

Halogen may be fluorine, chlorine, bromine or iodine.

The term Pseudohalogen as used herein represents a small monovalent electronegative group comprising 3 atoms or less. Examples of pesudohalogens are cyanide, cyanate or thiocyanate.

The term “palladium precursor” as used herein represents a palladium source capable of generating a catalytic system in combination with a suitable ligand and base.

For the A radical, optionally substituted C₄-C₂₄-arylsulphonyl is preferably trifluoromethanesulphonyl (triflate) or p-toluenesulphonyl.

In the context of the invention, all radical definitions, parameters and illustrations above and listed below, in general or specified within areas of preference, i.e. also between the particular areas and areas of preference, may be combined as desired.

In the context of the invention, “optionally substituted” when referring to groups comprised in the definitions of above-specified A, R, R¹ to R⁶ and R^{x} to R^{z} means that these groups may optionally bear one or more identical or different substituents.

Possible substituents included in the groups comprised in the definitions of A, R, R¹ to R⁶ and R^{x} to R^{z} include numerous organic groups, for example C₁-C₁₈-alkyl, cycloalkyl, aryl, C₁-C₁₈-alkoxy, C₁-C₁₈-haloalkoxy, in particular C₁-C₁₈-perfluoroalkoxy, halogen, C₁-C₁₈-haloalkyl, in particular partly fluorinated or perfluorinated C₁-C₁₈-alkyl, disubstituted amine, disubstituted phosphine, ether, thioether, disulphide, sulphoxide, sulphonic acid, sulphonate, aldehyde, keto, carboxylic ester, carbonyl chloride, carbonate, carboxylate, cyano, nitro, amino, hydroxyl, C₁-C₁₈-alkylsilane, C₁-C₁₈-alkoxysilane or carboxamide groups.

Palladium precursors used with preference are palladium (II) acetate, trisdibenzylideneacetonepalladium(0), allylpalladium(II) chloride dimer, palladium(II) chloride, palladium(II) acetylacetonate or palladium(II) nitrate. Particular preference is given to trisdibenzylideneacetonepalladium(0).

Preferred ligand(s) are mono- and bidentate phosphorus compounds, more preferably monodentate phosphines which bear a substituted biphenyl radical. Very particular preference is given to 2,4,6-triisopropyl-2′-biscyclohexylphosphinobiphenyl.

The base(s) used are preferably alkali metal or alkaline earth metal phosphates, for example potassium phosphate, alkali metal or alkaline earth metal carbonates, for example caesium carbonate, alkali metal or alkaline earth metal hydroxides, for example sodium hydroxide or potassium hydroxide, or alkali metal C₁-C₁₈-alkoxides, for example sodium tert-butoxide or potassium tert-butoxide, sodium methoxide or sodium tert-amylate.

The performance of the process according to the invention under protective gas atmosphere, for example nitrogen or argon atmosphere, may be advantageous but is not necessarily required.

The process according to the invention may be carried out at standard, elevated or reduced pressure, for example in the range from 0.5 to 50 bar. In general, it is carried out at standard pressure.

The reaction of the compounds of the general formula (II) with the compounds of the general formula (III) is carried out preferably at temperatures of 50° C. to 140° C., more preferably of 85° C. to 130° C., most preferably of 90° C. to 120° C. The reaction time at this temperature is preferably several hours, more preferably 0.2 to 36 h, most preferably 1 to 24 h.

The compounds of the general formula (II) and (III) and also the individual components of the catalyst system and of the solvent system may be combined in any sequence. This can be done in such a way that, in each case, the compounds of the general formula (II) and (III) are premixed in a portion of the solvent system, for example in a quantitative proportion or in a portion of the solvent components, and, independently thereof, the catalyst system in the remaining quantitative portion or the residual component(s) of the solvent system, and subsequently combined. In this case, either the catalyst system can be added to the solution of the compounds of the general formula (II) and (III) or vice versa.

The process according to the invention can be performed, for example, in such a way that the compounds of the general formula (II) and (III) are first initially charged in the solvent system or a portion of the solvent system, if appropriate at a lower temperature than the reaction temperature, preferably at room temperature, and optionally degassed, whereupon the catalyst system comprising the palladium precursor, ligand and base components, which have been combined in any sequence, if appropriate at a lower temperature than the reaction temperature, preferably at room temperature, if appropriate in the remaining portion of the solvent system, if appropriate with degassing and stirring, is added to the initially charged solution and the reaction mixture is subsequently brought to the desired reaction temperature and reacted for the appropriate reaction time. The reaction product can be removed from the reaction solution, for example, by filtration before or after cooling. If appropriate, one or more further customary purification step(s) may follow. Recycling of the catalyst system is possible.

The process according to the invention may be carried out either continuously or discontinuously, for example batchwise.

The process according to the invention is outstandingly suitable for the preparation of compounds of the general formula (I) which are, for example, important starting materials for the preparation of active ingredients for pharmaceutical, agrochemical or specialty chemistry applications. The process according to the invention allows the compounds of the general formula (I) to be prepared with an increased conversion compared to known processes.

The present invention further provides for the use of monodentate phosphines which bear a substituted biphenyl radical for preparing compounds of the general formula (I).

In a preferred embodiment, the ligand used is 2,4,6-triisopropyl-2′-biscyclohexylphosphinobiphenyl.

The process according to the present invention may be particularly suitable for the preparation of synthetic intermediates useful for the synthesis of pharmaceutical active ingredients, for example pharmaceutical active ingredients described in any of the following international patent applications WO 2004/000846, WO 2004/113347 and/or WO 2004/113349.

Particularly the process according to the present invention is suitable for the preparation of 3-(3-methylphenylamino)-6-oxo-7-phenyl-6,7-dihydrothieno[2,3-b]pyridine-2-nitrile.

### EXAMPLE

200.0 g (748 mmol) of 3-amino-6-oxo-7-phenyl-6,7-dihydrothieno[2,3-b]pyridine-2-nitrile, 2523 ml of tri-n-butylamine, 631 ml of 1,2-dimethoxyethane, 154.0 g of 3-bromotoluene (901 mmol), 239.0 g (1127 mmol) of potassium phosphate and 31.7 ml of water were initially charged and degassed by a cycle, carried out three times, of evacuation and aeration with argon. In a separate initial charge vessel, 300 ml of tetrahydrofuran were degassed by the above-described technique. 13.74 g (15 mmol) of trisdibenzylideneacetonepalladium(0) were added to this solvent. The suspension was degassed again and then stirred at 25° C. for 5 min. 14.31 g (30 mmol) of 2,4,6-triisopropyl-2′-biscyclohexylphosphinobiphenyl were then added and the suspension was stirred at 25° C. for 10 min. The stirred suspension was introduced into the reaction mixture through a cannula and the vessel was flushed with 88 ml of degassed tetrahydrofuran. The mixture was heated to an internal temperature of 96° C. with stirring and stirred at this temperature for 16 h. The reaction mixture was then cooled to 25° C. and filtered through a G3 frit. The solid which had been filtered off was added to a solution of 75.1 g of the disodium salt of ethylenediaminetetraacetic acid in 1922 ml of water and stirred at 25° C. for 2 h. The suspension was filtered off with suction through a G3 frit and washed twice with 300 ml of methanol. The filtercake thus obtained is dried at 50° C. and 200 mbar over 10 h. 232.7 g (87% of theory, GC: 97.8 area %) of 3-(3-methylphenylamino)-6-oxo-7-phenyl-6,7-dihydrothieno[2,3-b]pyridine-2-nitrile are obtained.

1. A process for preparing compounds of the general formula (I) 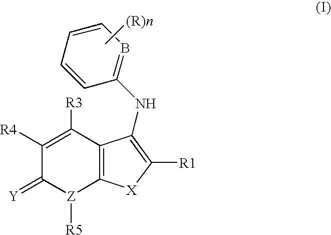
in which
X is O, S, NH or CH₂,
Y is O or S,
Z is N or CH,
R¹, R³, R⁴ are each independently H, optionally substituted C₁-C₁₈-alkyl, optionally substituted C₁-C₁₈-alkoxy, optionally substituted C₄-C₂₄-aryl, optionally substituted C₅-C₁₈-arylalkyl, halogen, optionally substituted C₁-C₁₈-alkylcarbonyl, optionally substituted C₄-C₂₄-arylcarbonyl, —COOR⁷, pseudohalogen, OH, NO₂, COR^{x} in which R^{x} is OH, OR^{y}, NR^{y}R^{z}, or NHet, where Ry and R^{z} are each independently optionally substituted C₁-C₁₈-alkyl, optionally substituted C₄-C₂₄-aryl or optionally substituted C₅-C₁₈-arylalkyl, and NHet is an optionally substituted 4- to 6-membered heterocycloalkyl group attached through a nitrogen atom to the group CO,
R⁵ is H, optionally substituted C₁-C₁₈-alkyl, optionally substituted C₄-C₂₄-aryl or optionally substituted C₅-C₁₈-arylalkyl,
R⁷ is an optionally substituted aryloxyaryl, alkanoyloxyalkyl or aroyloxyalkyl,
R is each independently H, optionally substituted C₁-C₁₈-alkyl, preferably C₁-C₆-alkyl, optionally substituted C₁-C₁₈-alkoxy, preferably C₁-C₆-alkoxy, optionally substituted C₄-C₂₄-aryl, preferably C₆-C₂₄-aryl, optionally substituted C₅-C₁₈-arylalkyl, halogen preferably fluorine, pseudohalogen, OH, NO₂ or COR^{x} in which R^{x} is OH, OR^{y} or NR^{y}R^{z}, where R^{y} and R^{z} are each independently optionally substituted C₁-C₁₈-alkyl, preferably C₁-C₆-alkyl, optionally substituted C₄-C₂₄-aryl, preferably C₆-C₂₄-aryl, or optionally substituted C₅-C₁₈-arylalkyl,
B is CH or N, and
n is 0 or an integer of 1 to 5,
by reacting compounds of the general formula (II) 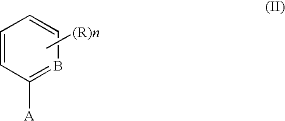
in which
A is halogen or —OR⁶ in which R⁶ is optionally substituted C₁-C₁₈-alkylsulphonyl or optionally substituted C₄-C₂₄-arylsulphonyl,
R, B and n are each as defined for the general formula (I),
with compounds of the general formula (III) 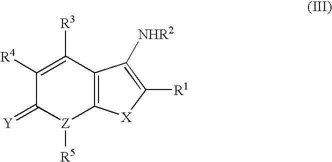
in which
R² is H, optionally substituted C₁-C₁₈-alkyl, optionally substituted C₄-C₂₄-aryl, or optionally substituted C₅-C₁₈-arylalkyl, and
R¹, R³ to R⁵, X, Y and Z are each as defined for the general formula (I),
in the presence of a catalyst system comprising at least one palladium precursor, at least one ligand and at least one base, characterized in that the reaction is carried out in a solvent system comprising at least two solvents selected from ethers, cyclic ethers, tertiary amines, aromatic hydrocarbons, alcohols and water.

2. The process according to claim 1, wherein the solvent system comprises at least two solvents selected from tetrahydrofuran, 1,2-dimethoxyethane, tri-n-butylamine and water.

3. The process according to claim 1, wherein the solvent system comprises more than two solvents selected from ethers, cyclic ethers, tertiary amines, aromatic hydrocarbons, alcohols and water.

4. The process according to claim 1, wherein the solvent system comprises tri-n-butylamine, 1,2-dimethoxyethane, tetrahydrofuran and water.

5. The process according to claim 1, wherein the palladium precursor used is palladium(II) acetate, trisdibenzylideneacetonepalladium(0), allylpalladium(II) chloride dimer, palladium(II) chloride, palladium(II) acetylacetonate or palladium(II) nitrate.

6. The process according to claim 1, wherein the ligand(s) used are mono- and bidentate phosphorus compounds.

7. The process according to claim 1, wherein the ligand(s) used are monodentate phosphines which bear a substituted biphenyl radical.

8. The process according to claim 1, wherein the base(s) used are alkali metal or alkaline earth metal phosphates, alkali metal or alkaline earth metal carbonates, alkali metal or alkaline earth metal hydroxides or alkali metal C₁-C₁₈-alkoxides.

9. The process according to claim 1 for the preparation of 3-(3-methylphenylamino)-6-oxo-7-phenyl-6,7-dihydrothieno[2,3-b]pyridine-2-nitrile.

10. (canceled)

11. (canceled)

12. In a method of conducting a chemical reaction in which a carbon is coupled to a nitrogen the improvement comprising conducting the reaction in a solvent system comprising at least two solvents selected from ethers, cyclic ethers, tertiary amines, aromatic hydrocarbons, alcohols and water.

13. The method of claim 12 wherein the reaction is a Pd-catalyzed reaction between an aryl halide or an aryloxysulyphonyl compound and an amine to form a C—N bond.
